(19) United States
(12) Patent Application Publication
(10) Pub. No.: US 2025/0379461 A1
(43) Pub. Date: Dec. 11, 2025

(54) AN INTRAORAL SCANNER BATTERY CHARGER

(71) Applicant: 3SHAPE A/S, Copenhagen K (DK)

(72) Inventors: Christian DÜNWEBER, Copenhagen K (DK); Edward MATOS, Copenhagen K (DK)

(73) Assignee: 3SHAPE A/S, Copenhagen K (DK)

(21) Appl. No.: 18/876,995

(22) PCT Filed: Jun. 20, 2023
(86) PCT No.: PCT/EP2023/066650

§ 371 Date: Dec. 19, 2024

(30) Foreign Application Priority Data
Jun. 20, 2022 (EP) 22179828.3

(51) Int. Cl. H02J 7/00; A61C 9/00

(52) U.S. Cl. CPC H02J 7/0044; A61C 9/0053; H02J 7/00045; H02J 7/0013; H02J 7/00712; H02J 7/007192; A61C 2204/002

(57) ABSTRACT

The disclosure relates to an intraoral scanner battery charger that includes two or more battery slots configured to receive an intraoral scanner battery, wherein each of the two or more battery slots includes a charging interface that is configured to an intraoral scanner battery interface of an intraoral scanner. The charging interface is configured to transfer a charging current to the intraoral scanner battery. The charger further includes a processor unit configured to control the charging current based on a prioritized charging algorithm. The prioritized charging algorithm includes transferring a first charging current to a first intraoral scanner battery and a second charging current to a second intraoral scanner battery, and wherein the first charging current is higher than the second charging current during a charging period, and during a subsequent charging period, the first charging current is lower than the second charging current.

### FIELD

The disclosure relates to an intraoral scanner battery charger, and more specially, to a charging algorithm that is configured to prioritize charging of two or more intraoral scanner battery so that at least one of the two batteries reach as fast a possible to a state of charge that is enough for allowing an intraoral scanner to perform at least a single complete scan of a patient's mouth, i.e. an oral cavity, with that battery.

### BACKGROUND

An intraoral scanner that is configured to communicate wireless with an external device is known to be powered by a rechargeable battery. This is convenient for the use as no cable between the scanner and the external device would intervene the user during a scanning session of a patient. Furthermore, the user is not limited by the length of the cable or obstacles that may collide with the cable. However, having the scanner powered by cable would mean that the user would not risk having a scanner that is not able to scan due to lack of power. One way of solving this while having a rechargeable battery powering the intraoral scanner is to have one or more extra batteries that are being charged while scanning a patient or when not using the scanner. Thereby, a battery may be ready to be used for a complete scan session when the charge capacity of the battery in use is no more suitable to perform a scan. Unfortunately, in some situations the user may forget to place the battery(ies) in the charger after being used, and that may result in a situation where the user has no batteries that are ready to be used for at least a complete scan session of a patient. In today's charger for an intraoral scanner battery, the batteries are being charged simultaneously with equal amount of charge power. Unfortunately, that results in a long and inconvenient charging period for charging a battery that is ready to be used for a whole scan session of a patient.

Therefore, there is a need for improving the charging period of an intraoral scanner battery such that it does not become inconvenient for the user to scan a patient with a wireless intraoral scanner powered by a rechargeable battery.

### SUMMARY

An aspect of the present disclosure is to provide a fast and reliable intraoral scanner battery charger system.

A further aspect of the present disclosure is to provide faster an intraoral scanner battery that is ready to be used for a complete intraoral scan of a patient's oral cavity, such as the teeth of the patient.

According to the aspects, an intraoral scanner battery charger system for charging an intraoral scanner battery that is configured to be detachable mounted to a handheld intraoral scanner. The system may comprise a handheld intraoral scanner that may be configured to perform 3D scanning, an intraoral scanner battery that is configured to be inserted into the handheld intraoral scanner and power the scanner. The system further comprises an intraoral scanner battery charger that may be configured to charge the intraoral scanner battery The intraoral scanner battery charger may comprise two or more battery slots that may be configured to receive an intraoral scanner battery. Each of the two or more battery slots may include a charging interface that is configured to an intraoral scanner battery interface of an intraoral scanner. The intraoral scanner may be configured to provide 2D images and/or 3D images to an external device of a mouth of a patient. The intraoral scanner is configured to scan the mouth by use of a projector unit, an image sensor and an image processor unit. The projector unit may include one or more light sources configured to emit light onto a dental object, and the image sensor may be configured to capture reflected light of the dental object. The image processor may then be configured to process the captured reflected light into raw image data, 2D image data, and/or 3D image data. The intraoral scanner battery charger may further comprise a processor unit configured to control the charging current based on a prioritized charging algorithm.

The prioritized charging algorithm may include transferring a first charging current to a first intraoral scanner battery and a second charging current to a second intraoral scanner battery, and the first charging current may be higher than the second charging current during a charging period, and during a subsequent charging period, the first charging current may be lower than the second charging current.

The prioritized charging algorithm may include prioritizing a charging of a first intraoral scanner battery higher than a charging of a second intraoral scanner battery during a charging period, and during a second charging period, the prioritized charging algorithm includes prioritizing a charging of a first intraoral scanner battery lower than a charging of a second intraoral scanner battery during a subsequent charging period.

The processor unit may be configured to assign a first intraoral scanner battery arranged in one of the two or more battery slots a master role, and to assign a second intraoral scanner battery a slave role. The prioritized charging algorithm may include charging the first intraoral scanner battery at a higher charging rate than the second intraoral scanner battery during a first charging phase, and during a second charging phase the charging rate of the first intraoral scanner battery is lower than the second intraoral scanner battery.

In each of the examples of the prioritized charging algorithm, the first intraoral scanner battery or the master intraoral scanner battery are not fully charged during the first charging phase, but instead, the first intraoral scanner battery or the master intraoral scanner battery is charged to an extend that the battery can be used for at least one full scan of a patient's mouth. Therefore, during the charging period none of the batteries being charged by the charger are fully charged. During the subsequent charging period at least one of the batteries would be fully charged.

The intraoral scanner battery charger may comprise an internal power supply. A maximum output current of the chargers internal power supply will be exceed if two intraoral scanner battery were to be charged at their maximum allowed rate simultaneously. The prioritized charging algorithm is configured to prioritize the charge current for the at least two battery slots, enabling the system to fast-charge one battery slot while capping the charge current of the other battery slot, to not exceed the maximum output current of the internal power supply while utilizing the full capacity, when needed. The charge current of the battery slot being prioritized highest, i.e. the master battery, will start to decrease when charging progresses. The algorithm directs the available current to the other battery slot, i.e. slave, being prioritized lower than the previous battery slot as it becomes available. The battery slot being populated first becomes the prioritization master.

The channel populated first becomes prioritization master. If plugged in simultaneously the highest state of charge (SOC) is prioritized highest. If equal SOC then a first battery slot is prioritized highest.

The master battery slot, i.e. the highest prioritized battery slot, is initialized with highest priority, which allows it to draw the maximum current one battery slot can deliver to the intraoral scanner battery. The slave battery slot is initialized with minimum priority, which caps the allowed current to a hardware set level where the sum of the master and slave is below the rating of the power supply by a reasonable margin. The algorithm continuously monitors the global current, that is available as an ADC input, and the priority of the slave is increased up to a target level global current is reached. At some point the master becomes fully charged (done) resulting in the slave getting maximum priority and the total current becomes dominated by the slave's current only. When the slave also becomes full, the total current is the 4harge4g4 current of the charger. Charging happens in two general phases, i.e. charging period, constant current (CC) up to a certain cell voltage whereafter constant voltage (CV) is used.

The intraoral scanner battery charger may include a memory configured to store information on a minimum charge capacity for an intraoral scanner battery, and wherein the minimum charge capacity of an intraoral scanner battery is enough for an intraoral scanner to perform a complete intraoral scan of a patient's mouth.

During a part of the charging period, the first charging current is equal to a target charging current and the second charging current is equal to a minimum charging current. Thereby, the first intraoral scanner battery is being charged fast while the second intraoral scanner battery is being charged slower than the first battery. This would result in a faster battery that is ready to be used by an intraoral scanner for scanning at least a complete scan of a patient's mouth.

During a part of the subsequent charging period, the first charging current is equal to a minimum charging current and the second intraoral scanner battery is equal to a target charging current. Thereby, the first intraoral scanner battery is ready to be used by an intraoral scanner for at least performing a single complete scan of a patient's mouth, and the second intraoral scanner battery is being faster charged for the purpose of faster reaching to the point where the battery is also ready to be used for at least a single complete scan of a patient's mouth.

The charger may be configured to provide a total charging current to at least one or more intraoral scan batteries, and the total charging current is a sum of the first and the second charging current, and wherein the total charging current is below a supply current with a safety margin.

The intraoral scanner battery charger may be configured to communicate wirelessly or wired to an intraoral scanner battery being inserted into one of the two or more battery slots. The communication may be performed via the charging interface, and the communication may be directionally or bidirectionally. The charger may include a wireless interface configured to communicate wirelessly with the intraoral scanner battery. The wireless interface may be based on amplitude modulation, phase modulation, or time modulation, or a combination thereof, or a Bluetooth communication protocol or a WIFI protocol.

The intraoral scanner battery charger may include a memory configured store a firmware update, and wherein the firmware update is transferred via the charging interface or the wireless interface.

Via the charging interface or the wireless interface, the charger may be configured to receive data from the battery or an external device, and wherein the data may be firmware, firmware update, settings, battery information, such as charging algorithm that relates to a battery type, a minimum charge capacity that corresponds to at least a single complete scan of a patient's mouth using an intraoral scanner etc.

An intraoral system may include the intraoral scanner battery charger, the intraoral scanner batter, and the intraoral scanner. The system is configured to update the charger via the intraoral scanner and the battery. For example, when the battery is inserted into the scanner a software update package, a firmware update package, or a setting update package may be transferred from an external server to the scanner wirelessly or wired, and the package is then forwarded to the battery. When the battery is then placed into a battery slot, the package is then automatically forwarded to the charger via the charging interface or the wireless interface. In another example, the charger may be connected to an external server wirelessly or wired, and the charger may receive a software update package, a firmware update package, or a setting update package to be forwarded to the battery. The package may be for the battery, and in this example, the battery is configured to install the package into a part of a memory of the battery. In another example, the battery forwards the package to the scanner which then installs the package into a part of the memory of the scanner.

During another part of the charging period, a first charging current to the first intraoral scanner battery may be reduced gradually while a second charging current to the second intraoral scanner battery is increased gradually. The gradually reduction and increase may be performed simultaneously for obtaining an almost constant total charging current. The sum between the gradually decreasing first charging current and the gradually increasing second charging current is constant or about constant. Thereby, an optimal transfer of the batteries is obtained as the maximum total charging current is always utilized for charging batteries when charging of the batteries are needed.

The intraoral scanner battery charger may comprise a temperature sensor configured to measure a temperature external to the intraoral scanner battery. The temperature may be within a housing of the charger. The processor unit may then be configured to monitor the temperature for controlling the charging current to the first intraoral scanner battery and/or the second intraoral scanner battery. Thereby, it is avoided to overheat the charger which may damage the charger. The processor unit may be configured to correlate the measured temperature to a battery temperature which indicates a temperature of the battery. Thereby, the processor unit is configured to prevent the battery of being damaged due to overheating of the battery during charging. The correlation between the measured temperature external to the battery may be based on a battery correlation that is stored on the memory of the charger. The battery correlation may be determined during the manufacturing of or calibration of the charger.

The processor unit may be configured to increase the charging current to the second intraoral scanner battery if detecting that the first intraoral scanner battery is removed from a battery slot of the two or more battery slots. The increase of the charging rate of the charging current may be faster than the increase of the charging rate when going from the charging period to the subsequent charging period, i.e. the increase of the charging current of the second intraoral scanner battery during the transfer from a slave role to a master role of the second intraoral scanner battery.

The detection of the removed first Intraoral scanner battery and/or the second intraoral scanner battery may be based on an impedance measurement of the charging interface.

The battery slot may be a shaped as tube configured to receive a cylindrical shaped intraoral scanner battery.

For preventing a none authorized battery to be inserted into the battery slot of the intraoral scanner battery charger, the charger includes an authenticator unit configured to authenticate an intraoral scanner battery being inserted into each of the two or more battery slots. The authentication of the battery may be based on a common known secret key. The key may be common for both the intraoral scanner battery charger, at least an intraoral scanner battery and an intraoral scanner. The battery may be configured to be inserted into the charger and the scanner.

Each of the two or more battery slots includes a guide mean configured to guide the intraoral scanner battery for obtaining an optimal connection of the battery to the charging interface.

The processor may be configured to monitor the one or more battery slots within one or more prioritize periods. If for example, one of at least two batteries is removed from at least one of the two or more battery slots, the processor is configured to detect during a prioritize period whether a battery is removed or not. If the processor detect a battery has been removed, the processor is configured to immediately increase a charging current to the other battery which is arranged within one of two or more battery slots.

The target charging current may be between a maximum charging current and a delta charging current. The processor may be configured reduce the charging current if above the maximum charging current and/or increase the charging current if below the delta charging current.

The charger may include a processor configured to measure the total charging current and a Digital-Analog converter configured to convert the total charging current in steps. The slave current, i.e the second charging current, may then change in steps that will get it closest to the target current. The total charging current may be defined by a threshold and a neutral band (defined between the maximum charging current and the delta charging current) and based on knowledge of the hardware. The algorithm will increment or decrement the slav's current to keep the total charging current within the neutral band. When within the neutral zone, no adjustments are made.

If the battery of the master battery slot is removed, the battery of the slave battery slot becomes the new master in the subsequent charging period and gets full prioritization.

Should the maximum current of the internal power supply be exceeded, a hardware trip will occur that lowers the priority of both the master and slave channel in hardware. The firmware detects this and lowers the priority of the slave and then resets the hardware trip and continues the algorithm.

A further aspect of the disclosure is a method for charging intraoral scanner batteries, wherein the method comprising:
receiving an intraoral scanner battery in each of two or more battery slots,
transferring a charging current to the intraoral scanner battery,
controlling the charging current based on a prioritized charging algorithm, and
wherein the prioritized charging algorithm including:
   transferring a first charging current to a first intraoral scanner battery,
   transferring a second charging current to a second intraoral scanner battery, and
      wherein the first charging current is higher than the second charging current during a charging period, and during a subsequent charging period, the first charging current is lower than the second charging current.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIGS. 1A and 1B illustrate an intraoral scanner battery charger,

FIG. 2 illustrate a system including an intraoral scanner battery charger, an intraoral scanner battery, and an intraoral scanner,

FIG. 3 illustrates a prioritizing charging algorithm,

FIGS. 4A, 4B, and 4C illustrate an intraoral scanner battery charger,

FIG. 5 illustrates a prioritizing charging algorithm,

FIG. 6 illustrates an intraoral scanner battery charger,

FIG. 7 illustrates a prioritizing charging algorithm, and

FIG. 8 illustrates a prioritizing charging algorithm,

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intra-oral scan data may be performed by a dental scanning system that may include an intraoral scanner such as the TRIOS series scanners from 3Shape A/S. The dental scanning system may include a wireless capability as provided by a wireless network unit. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled during a scanning session, such that at least some sets of sub-scans overlap at least partially, in order to enable stitching in the post-processing. The result of stitching is the digital 3D representation of a surface larger than that which can be captured by a single sub-scan, i.e. which is larger than the field of view of the 3D scanning device. Stitching, also known as registration, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental object and a sequence of images of the different pattern configurations are acquired by one or more cameras located at an angle relative to the projector unit.

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono- or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit or a wired network unit. The wireless network unit is configured to wirelessly connect the dental scanning system to the network comprising the plurality of network elements including the at least one network element configured to receive the processed data. The wired network unit is configured to establish a wired connection between the dental scanning system and the network comprising the plurality of network elements including the at least one network element configured to receive the processed data.

The dental13harge13gng system preferably further comprises a processor configured to generate scan data intra-oral scan data by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance color images, x-ray images, and/or combinations thereof.

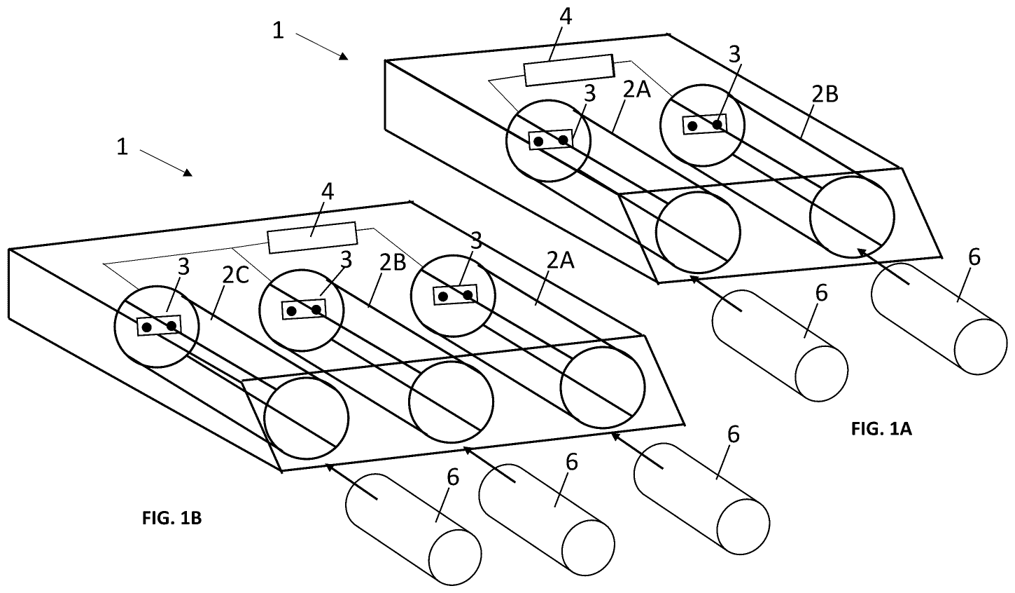

FIGS. 1A and 1B illustrate an intraoral scanner battery charger 1 configured to charge one or more intraoral scanner battery. The charger 1 comprises two or more battery slots (2A,2B,2C) configured to receive an intraoral scanner battery 6, wherein each of the two or more battery slots (2A,2B, 2C) includes a charging interface 3. The charging interface 3 is configured to an intraoral scanner battery interface of an intraoral scanner, and the charging interface 3 is configured to transfer a charging current to the intraoral scanner battery 6. The charger 1 includes a processor unit 4 configured to control the charging current based on a prioritized charging algorithm. FIG. 1A illustrates a charger 1 that includes two battery slots (2A,2B), and FIG. 1B illustrates a charger 1 that includes more than two battery slots. (2A,2B,2C).

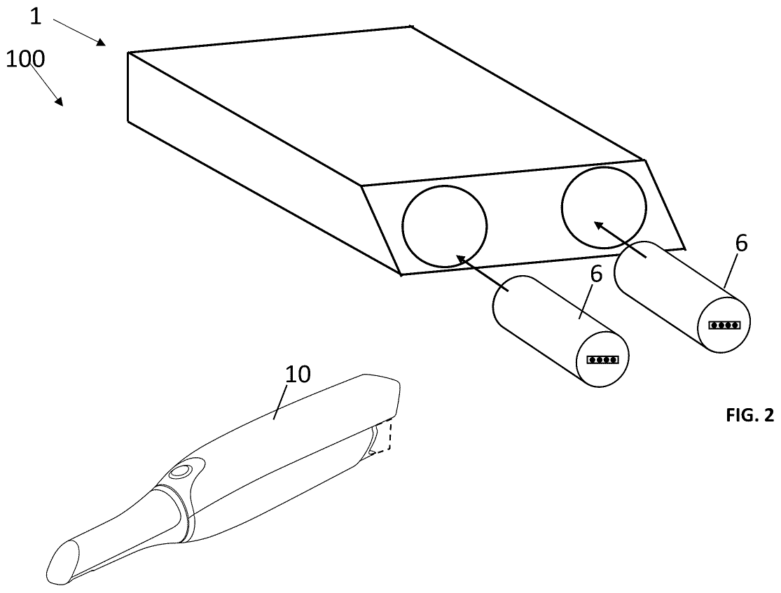

FIG. 2 illustrates a system 100 that includes the charger 1, at least an intraoral scanner battery 6 and an intraoral scanner 10. The charging interface is configured to an intraoral scanner battery interface 6 of the intraoral scanner 10. In this example, the battery 6 is configurable to be inserted into the charging interface 3 of the charger 1 and the battery interface of the scanner 10.

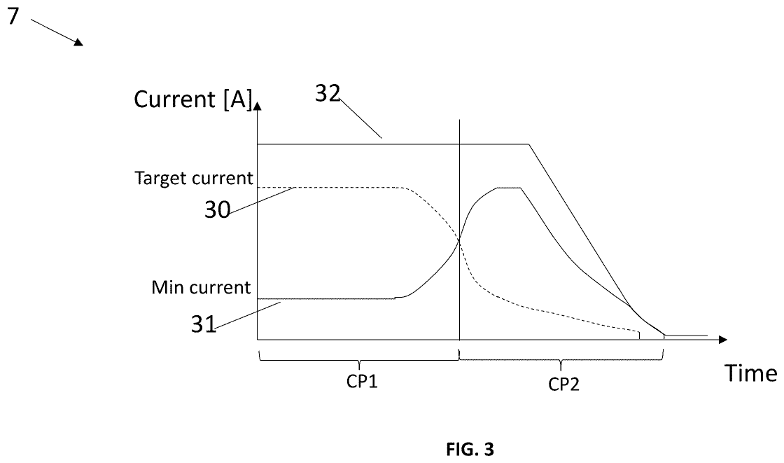

FIG. 3 illustrates the prioritized charging algorithm 7. The prioritized charging algorithm 7 includes transferring a first charging current 30 to a first intraoral scanner battery 6A and a second charging current 31 to a second intraoral scanner battery 6B, and wherein the first charging current 30 is higher than the second charging current 31 during a charging period CP1, and during a subsequent charging period CP2, the first charging current 30 is lower than the second charging current 31. During a part of the charging period CP1, the first charging current 30 is equal to a target charging current and the second charging current 31 is equal to a minimum charging current. During a part of the subsequent charging period CP2, the first charging current 30 is equal to a minimum charging current and the second intraoral scanner battery 31 is equal to a target charging current. A total charging current 32 is a sum of the first 30 and the second 3115hargeing current, and wherein the total charging current 32 is below a supply current with a safety margin. During another part of the charging period CP1, a first charging current 30 to the first intraoral scanner battery 6A is reduced gradually while a second charging current 31 to the second intraoral scanner battery 6B is increased gradually. The sum between the gradually decreasing first charging current 30 and the gradually increasing second charging current 31 is constant or about constant. The charging of each batteries 6 happens in two general phases; a constant current (CC) up to a certain cell voltage whereafter constant voltage (CV) is used. In the constant current phase, the charging current is either constant or increasing, and in the constant voltage phase, the charging current is reduced.

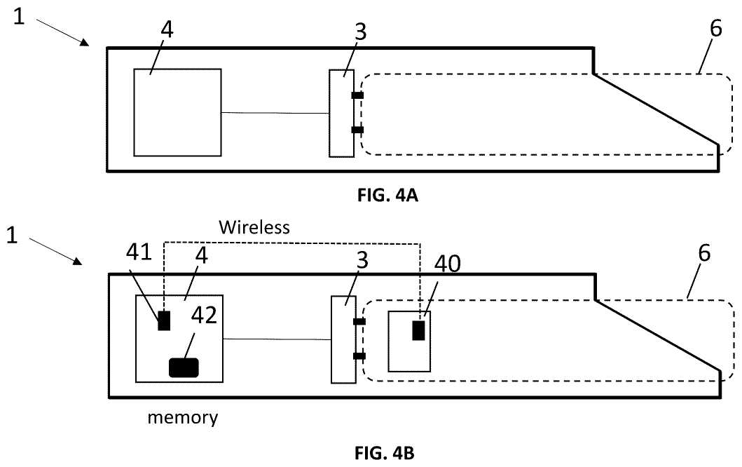

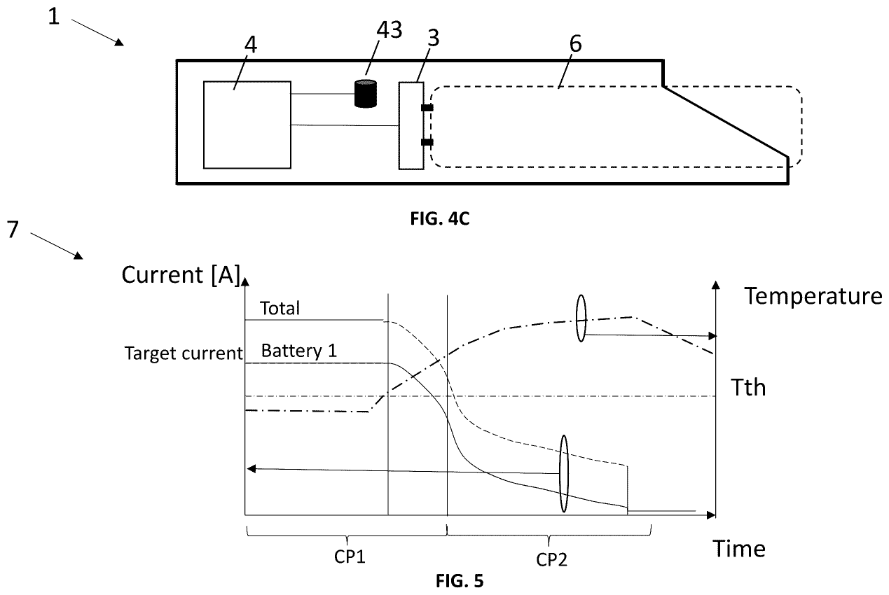

FIGS. 4A, 4B and 4C illustrates the charger 1 configured to communicate data directionally or bidirectionally to the intraoral scanner battery 6. In FIG. 4A, the communication is provided via the charging interface 3, i.e. the communication is performed via contacts, wires, or inductive. In FIG. 4B, the communication is provided via a wireless interface 41 to a wireless interface 40 of the battery 6. The charger 1 includes a memory 32 configured store a firmware update, and the firmware update is received via the charging interface 3 or the wireless interface 41. In FIG. 4C, the charger 1 includes a temperature sensor 43 configured to measure a temperature external to the intraoral scanner battery 6. The processor unit 4 is configured to decrease the charging current to the first intraoral scanner battery 6A and/or the second intraoral scanner battery 6B based on the measured temperature, see FIG. 5. In FIG. 5, the charging current is reduced as the measured temperature is above a temperature threshold, Tth, which indicates a temperature that is ideal for the charging of a battery.

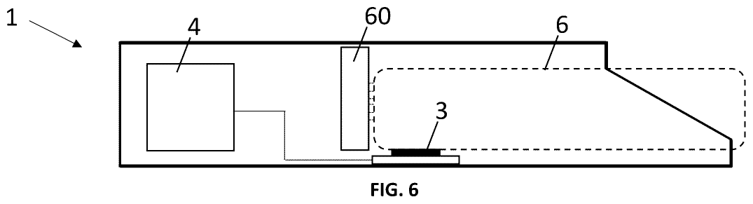

FIG. 6 illustrates an example of the charger where the battery slot (2A,2B,2C) includes at least a magnet for aligning the battery 6 for optimal connection to the battery interface 3.

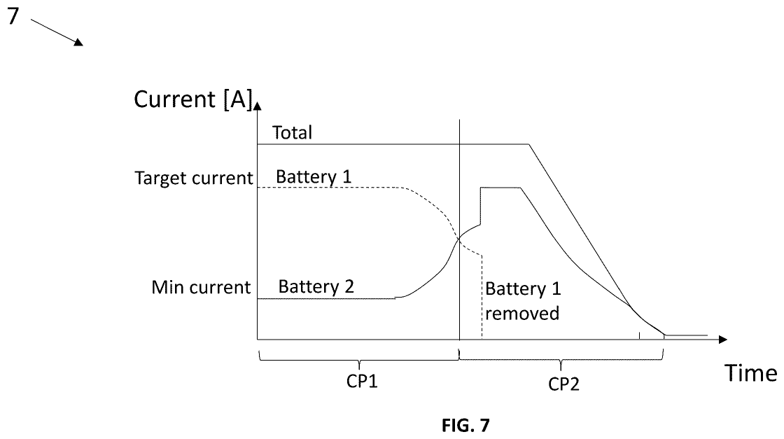

FIG. 7 illustrates an example wherein the processor unit 4 is configured to increase the charging current to the second intraoral scanner battery 6B when detecting that the first intraoral scanner battery 6A is removed from a battery slot (2A,2B,2C) of the two or more battery slots. The detection of the removed first intraoral scanner battery 6A and/or the second intraoral scanner battery 6B is based on an impedance measurement performed via for example the charging interface 3.

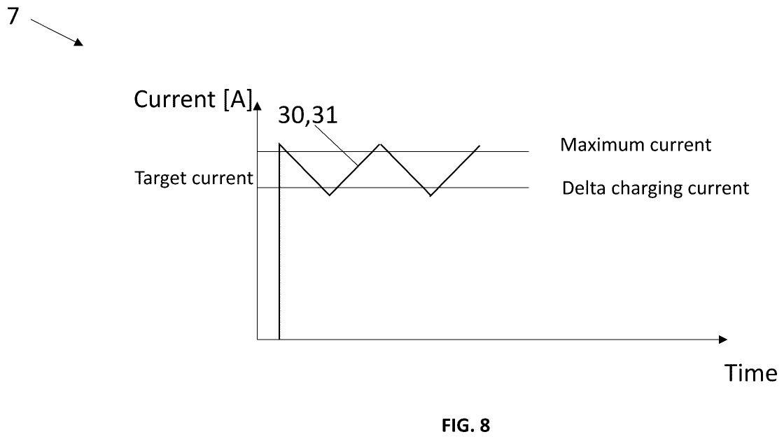

FIG. 8 illustrates that the target charging current (30,31) should be controlled to be between a maximum charging current and a delta charging current. The processor unit 4 is configured to reduce the charging current (30,31) if above the maximum charging current and/or increase the charging current if below the delta charging current.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/o” includes any and all combinations of one or more of the associated listed items. The steps of any disclosed method is not limited to the exact order stated herein, unless expressly stated otherwise.

It should be appreciated that reference throughout this specification to ““one embodimen”” or ““an embodimen”” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. An intraoral scanner battery charger system that is configured to charge an intraoral scanner battery of a handheld intraoral scanner, wherein the system comprising:
a handheld intraoral scanner that is configured to perform 3D scanning,
an intraoral scanner battery that is configured to be inserted into the handheld intraoral scanner and power the scanner,
an intraoral scanner battery charger that is configured to charge the intraoral scanner battery, and wherein the intraoral scanner battery charger includes:
two or more battery slots configured to receive the intraoral scanner battery, wherein each of the two or more battery slots includes a charging interface that is configured to an intraoral scanner battery interface of the handheld intraoral scanner, and the charging interface is configured to transfer a charging current to the intraoral scanner battery,
a processor unit configured to control the charging current based on a prioritized charging algorithm, and
wherein the prioritized charging algorithm includes transferring a first charging current to a first intraoral scanner battery of the system and a second charging current to a second intraoral scanner battery of the system, and wherein the first charging current is higher than the second charging current during a charging period, and during a subsequent charging period, the first charging current is lower than the second charging current.

2. The intraoral scanner battery charger system according to claim 1, wherein during a part of the charging period, the first charging current is equal to a target charging current and the second charging current is equal to a minimum charging current.

3. The intraoral scanner battery charger system according to claim 1, wherein the charging interface is configured to communicate data directionally or bidirectionally between the intraoral scanner battery and the intraoral scanner battery charger.

4. The intraoral scanner battery charger system according to claim 1, comprising a wireless interface configured to communicate with an intraoral scanner

5. The intraoral scanner battery charger system according to claim 3, comprising a memory unit configured store a firmware update, and wherein the firmware update is received via the charging interface or the wireless interface.

6. The intraoral scanner battery charger system according to claim 4, wherein the wireless interface includes a Bluetooth communication protocol or a WIFI protocol.

7. The intraoral scanner battery charger system according to claim 1, wherein during another part of the charging period, a first charging current to the first intraoral scanner battery is reduced gradually while a second charging current to the second intraoral scanner battery is increased gradually.

8. The intraoral scanner battery charger system according to claim 7, wherein the sum between the gradually decreasing first charging current and the gradually increasing second charging current is constant or about constant.

9. The intraoral scanner battery charger system according to claim 1, comprising a temperature sensor configured to measure a temperature external to the intraoral scanner battery.

10. The intraoral scanner battery charger system according to claim 9, wherein the processor unit is configured to decrease the charging current to the first intraoral scanner battery and/or the second intraoral scanner battery based on the measured temperature.

11. The intraoral scanner battery charger system according to claim 1, wherein the processor unit is configured to increase the charging current to the second intraoral scanner battery if detecting that the first intraoral scanner battery is removed from a battery slot of the two or more battery slots.

12. The intraoral scanner battery charger system according to claim 11, wherein the detection of the removed first intraoral scanner battery and/or the second intraoral scanner battery is based on an impedance measurement.

13. The intraoral scanner battery charger system according to claim 1, comprising an authenticator unit configured to authenticate an intraoral scanner battery being inserted into each of the two or more battery slots.

14. The intraoral scanner battery charger system according to claim 13, wherein the authentication of the battery is based on a common known secret key stored in a memory of the intraoral scanner battery charger.

15. The intraoral scanner battery charger system according to claim 14, wherein the common-known secret key is common for both the intraoral scanner battery charger, at least an intraoral scanner battery and an intraoral scanner, and wherein the battery is configured to be inserted into the charger and the scanner.

16. The intraoral scanner battery charger system according to claim 2, wherein the target charging current is between a maximum charging current and a delta charging current.

17. The intraoral scanner battery charger system according to claim 16, wherein the processor unit is configured to reduce the charging current if above the maximum charging current and/or increase the charging current if below the delta charging current.

18. A method for charging intraoral scanner batteries, wherein the method comprising:
receiving an intraoral scanner battery in each of two or more battery slots,
transferring a charging current to the intraoral scanner battery,
controlling the charging current based on a prioritized charging algorithm, and
wherein the prioritized charging algorithm including:
transferring a first charging current to a first intraoral scanner battery,
transferring a second charging current to a second intraoral scanner battery, and
wherein the first charging current is higher than the second charging current during a charging period, and during a subsequent charging period, the first charging current is lower than the second charging current.

19. An intraoral scanner system comprising at least an intraoral scanner battery, an intraoral scanner and an intraoral scanner battery charger according to claim 1.
